(19) United States
(12) Patent Application Publication
(10) Pub. No.: US 2018/0056034 A1
(43) Pub. Date: Mar. 1, 2018

(54) CENTRAL VENOUS CATHETER ACCESS ASPIRATION SYSTEM

(71) Applicant: Jacob Cynamon, Suffern, NY (US)

(72) Inventor: Jacob Cynamon, Suffern, NY (US)

(21) Appl. No.: 15/684,884

(22) Filed: Aug. 23, 2017

Related U.S. Application Data
(60) Provisional application No. 62/378,360, filed on Aug. 23, 2016.

(51) Int. Cl. A61M 25/00; A61B 5/1459

(52) U.S. Cl. CPC A61M 25/0029; A61M 25/0068; A61B 5/02152; A61M 2025/0031; A61B 5/1459

(57) ABSTRACT

A central venous catheter access aspiration system using a single connection is provided.

### CROSS-REFERENCE TO RELATED APPLICATION

This application claims the benefit of priority of U.S. provisional application No. 62/378,360, filed 23 Aug. 2016, the contents of which are herein incorporated by reference.

### BACKGROUND OF THE INVENTION

The present invention relates to medical devices and, more particularly, to a central venous catheter access aspiration system using a single connection.

A central venous catheter (CVC) is an intravenous access into the central, large-vein portion of a person's blood stream. A CVC provides a way of injecting medication or sampling blood. Drawing blood from a CVC, however, may lead to increased incidence of infections partially due to the current necessity of multiple syringe connections to obtain blood. Another issue is inadequate flush of the catheter, resulting in unacceptable residual blood. In fact, some institutions have mandated that CVCs not be used to draw blood samples. Unfortunately, a single attachment with one-way valves is not currently available for obtaining blood samples.

As can be seen, there is a need for a device with a single CVC-attachment and one-way valves embodying a method for aspirating of waste, aspirating of blood samples and forward flushing with a prefilled syringe with adequate fluid. Such a single safe connection system, with the ability to perform the entire blood sampling procedure without multiple connections and disconnections, is advantageous because the one-way valves prevents misuse, and a vacutainer connection enables needleless transfer of blood into vacutainer tubes, and a single connection will decrease infection and thrombosis rates.

### SUMMARY OF THE INVENTION

In one aspect of the present invention, a central venous catheter access aspiration device includes a hub connector fluidly connected to a proximate end of each of a first, second and third tube; a first, second, and third one-way valve assembly disposed in each respective tube between each proximate and distal end thereof; the first and third one-way valve assembly being distal directional; and the second one-way valve assembly being proximate directional.

In another aspect of the present invention, the central venous catheter access aspiration device includes a hub connector fluidly connected to a proximate end of each of a first, second and third tube; a first, second, and third syringe fluidly connected to a distal end of first, second and third tube respectively, wherein each one-way valve assembly is pressure actuable via each operatively associated syringe by either aspirating back into the syringe or flushing into the central venous catheter to selectively interconnect the distal and proximate ends of each respective tube; a first, second, and third one-way valve assembly disposed in each respective tube between each proximate and distal end thereof; a fourth tube fluidly connected to the third tube between the distal end of the third tube and the third one-way valve assembly; a fourth one-way valve assembly disposed in the fourth tube; a vacutainer connection provided on the distal end of the fourth tube; the first, third and fourth one-way valve assembly being distal directional; and the second one-way valve assembly being proximate directional.

In yet another aspect of the present invention, the method of aspirating of waste, aspirating of blood samples and forward flushing of a central venous catheter utilizing only a single connection thereto, includes the steps of: attaching the hub connector of the central venous catheter access aspiration device to the central venous catheter; aspirating residual fluid from said catheter via the operatively associated the first syringe; aspirating a predetermined sample from said catheter via the operatively associated the third syringe; and flushing said catheter using a predetermined fluid provided in the second syringe via the operatively associated the second syringe. Some embodiments may include the additional step of aspirating a sample portion of the predetermined sample through the fourth tube via the operatively associated vacutainer connection.

These and other features, aspects and advantages of the present invention will become better understood with reference to the following drawings, description and claims.

### BRIEF DESCRIPTION OF THE DRAWINGS

FIG. 1 is an elevation view of an exemplary embodiment of a hub connector of the present invention; and

FIG. 2 is a schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a central venous catheter access aspiration system using a single connection.

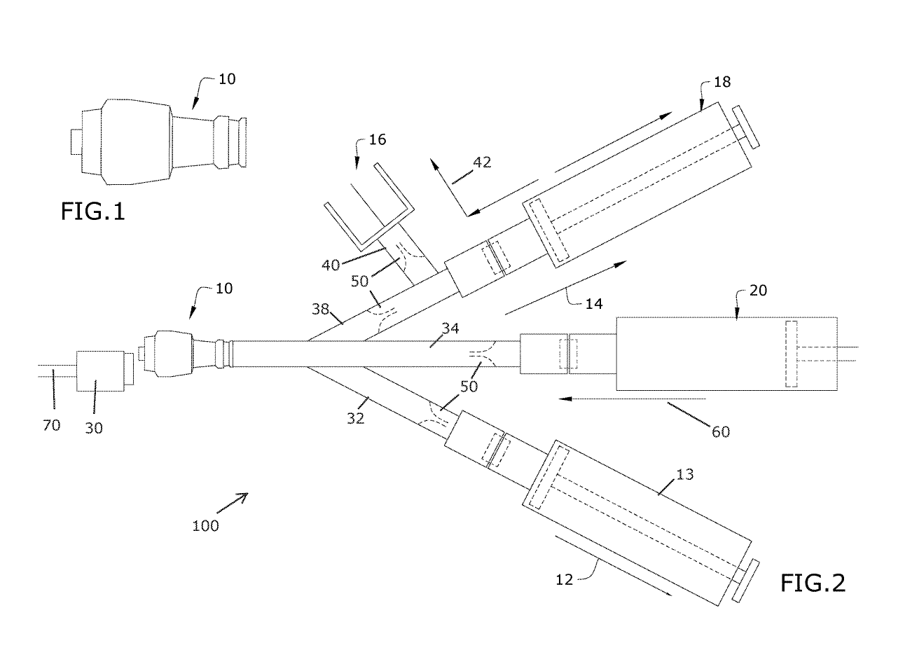

Referring to FIGS. 1 and 2, the present invention may include a CVC access aspiration system 100 for attaching to a CVC 70 via dead-ender hub 30. In some embodiments, there may be no hub 30, though the present invention can fluidly connect the plurality of tubes to the CVC 70 nonetheless. The CVC access aspiration system 100 may provide a hub connector 10 for removably connecting to the dead-ender hub 30 or CVC 70. The hub connector 10 fluidly communicates to a proximate end of a first tube 32, a second tube 34, and a third tube 38. Between the proximate end and a distal end of each tube may be a one-way valve assembly 50. Referring to FIG. 2, the one-way valve assembly 50 of the first and third tubes 32 and 38 are adapted to allow fluid to flow only in a distal direction: from the proximate end to the distal end (from the catheter 70 into the syringe). The one-way valve assembly 50 of the second tube is oriented to allow fluid to flow only in a proximate direction: from the distal end to the proximate end (from the syringe into the catheter 70). Fluidly connected to the distal end of each tube 32, 34, and 38 is a waste syringe 13, a flush syringe 20, and a sampler syringe 18, respectively. Each valve assembly 50 is actuable by pressure, either by aspiration or by flushing, to selectively and fluidly connect between an operatively associated syringe via the associated tube.

Disposed between the one-way valve assembly 50 of the third tube 38 and the distal end of the third tube 38/sampler syringe 18 interface is a proximate end of a fluidly connected fourth tube 40, wherein the fourth tube 40 provides a distal directional one-way valve assembly 50. Operatively connected to the distal end of the fourth tube 40 may be a vacutainer connection 16.

A method of using the present invention may include the following. The CVC access aspiration system 100 disclosed above may be provided. After cleaning the CVC 70 access port and/or the dead-ender hub 30, a user may attach the CVC access aspiration system 100 via to the hub connector 10 thereto. The user may aspirate residual fluid 12 from the catheter 70 via the operatively associated waste syringe 13. Then the user may aspirate a predetermined blood sample 14 via the operatively associated sample syringe 18. Then the user may flush the catheter 70 using a predetermined fluid via the operatively associated flush syringe 20. Then via the fourth tube 40, the user may aspirate a sample portion 42 of the predetermined blood sample 14 into an operatively associated vacutainer tube (not shown) via the vacutainer connection 16. Then the user may remove and discard the CVC access aspiration system 100. Thereby, the one single connection with built-in preventive measures of the present invention eliminates potential introduction of infection or catheter thrombosis. Furthermore, no needles would be required.

In certain embodiments, a similar device can also be used to administer drugs or fluid, via a proximate directional one-way valve assembly 50 disposed in the third tube 38 (from the syringe to the catheter 70), then the line is flushed with the pre-filled fluid 60.

In an alternative embodiment, a vacutainer device (not shown) can be fluidly attached directly to the sample syringe 18 to draw the blood into a vacutainer tube, and so the fourth tube 40 would not be required.

In all embodiments, the present invention eliminates steps and connections each time the central catheter 70 is accessed, as well as enables a smaller, user-friendly medical device.

It should be understood, of course, that the foregoing relates to exemplary embodiments of the invention and that modifications may be made without departing from the spirit and scope of the invention as set forth in the following claims.

What is claimed is:

1. A device, comprising:
a hub connector fluidly connected to a proximate end of each of a first, second and third tube;
a first, second, and third one-way valve assembly disposed in each respective tube between each proximate and distal end thereof;
the first and third one-way valve assembly being distal directional; and
the second one-way valve assembly being proximate directional.

2. The device of claim 1, further comprising a first, second, and third syringe fluidly connected to a distal end of first, second and third tube respectively.

3. The device of claim 2, wherein each one-way valve assembly is pressure actuable via each operatively associated syringe to selectively interconnect the distal and proximate ends of each respective tube.

4. The device of claim 1, further comprising a fourth tube fluidly connected to the third tube between the distal end of the third tube and the third one-way valve assembly; and a fourth one-way valve assembly disposed in the fourth tube in a distal directional orientation.

5. The device of claim 4, further comprising a vacutainer connection provided on the distal end of the fourth tube.

6. A device, comprising:
a hub connector fluidly connected to a proximate end of each of a first, second and third tube;
a first, second, and third syringe fluidly connected to a distal end of first, second and third tube respectively, wherein each one-way valve assembly is actuable by pressure via each operatively associated syringe to selectively interconnect the distal and proximate ends of each respective tube;
a first, second, and third one-way valve assembly disposed in each respective tube between each proximate and distal end thereof;
a fourth tube fluidly connected to the third tube between the distal end of the third tube and the third one-way valve assembly;
a fourth one-way valve assembly disposed in the fourth tube;
a vacutainer connection provided on the distal end of the fourth tube;
the first, third and fourth one-way valve assembly being distal directional; and
the second one-way valve assembly being proximate directional.

7. A method of aspirating of waste, aspirating of blood samples and forward flushing of a central venous catheter utilizing only a single connection thereto, comprising the steps of:
attaching the hub connector of the device of claim 1 to the central venous catheter;
aspirating residual fluid from said catheter via the operatively associated the first syringe;
aspirating a predetermined sample from said catheter via the operatively associated the third syringe; and
flushing said catheter using a predetermined fluid provided in the second syringe via the operatively associated the second syringe.

8. A method of aspirating of waste, aspirating of blood samples and forward flushing of a central venous catheter utilizing only a single connection thereto, comprising the steps of:
attaching the hub connector of the device of claim 6 to the central venous catheter;
aspirating residual fluid from said catheter via the operatively associated the first syringe;
aspirating a predetermined sample from said catheter via the operatively associated the third syringe; and
flushing said catheter using a predetermined fluid provided in the second syringe via the operatively associated the second syringe.

9. The method of claim 8, further comprising the step of aspirating a sample portion of the predetermined sample through the fourth tube via the operatively associated vacutainer connection.
